(19) United States
(12) Patent Application Publication
(10) Pub. No.: US 2007/0292543 A1
(43) Pub. Date: Dec. 20, 2007

(54) Extracts From Roots And/Or Shoots Of Asparagus Officinalis L., Their Preparation And Their Use

(75) Inventor: Uwe Schnee, Murnau (DE)

Correspondence Address: GREENBLUM & BERNSTEIN, P.L.C., 1950 ROLAND CLARKE PLACE, RESTON, VA, 20191, US

(73) Assignee: PLANTINA ENTWICKLUNGS-GMBH, Muenchen (DE)

(21) Appl. No.: 11/575,591

(22) PCT Filed: Sep. 22, 2005
(86) PCT No.: PCT/EP05/10282

§ 371 Date: Jul. 31, 2007

(30) Foreign Application Priority Data
Sep. 22, 2004 (EP) 04022522.9

(51) Int. Cl. A61K 36/8965; A61K 125/00; A61P 7/10; A61K 135/00

(52) U.S. Cl. 424/773; 424/779

(57) ABSTRACT

The present invention relates to a saponine-depleted raw extract and to a saponine-free extract from roots and/or shoots of Asparagus Officinalis L. The invention also relates to the preparation of saponine-depleted raw extracts and saponine-free extracts from roots and/or shoots of Asparagus Officinalis L. and to their use for the manufacture of a medicament.

The present invention relates to a saponine-depleted raw extract and to a saponine-free extract from roots and/or shoots of Asparagus Officinalis L. The invention also relates to the preparation of saponine-depleted raw extracts and saponine-free extracts from roots and/or shoots of Asparagus Officinalis L. and to their use for the manufacture of a medicament.

Asparagus Officinalis L. is traditionally cultivated in a temperate climate as food and medicinal plant for at least two thousand years. There are two types of asparagus produced in the world: White asparagus in which the shoots, which are young stems, are harvested from raised beds just when appearing on the soil; and green asparagus in which the shoots are harvested at the ground level.

The underground parts, i.e. the asparagus roots, are traditionally used as a diuretic in the dropsy disease. This diuretic property has been recommended to be made use of by ancient authors as Dioscoridos, Plinius, Celsius, and others. In the phytotherapy Asparagus roots are used in the form of cut rhizomes for teas as well as preparations having other galenical forms for internal use, for example in the form of a dried powder. The fields of application range from the irrigation therapy for inflammatory diseases of the urinary tract to the prevention of renal gravel and kidney stones, respectively. In addition, the powdered Asparagus roots are helpful in the weight reduction through augmented aquarese and as concomitant therapy in treating light essential hypertension. Up to now, any therapeutic effect of Asparagus shoots is unknown.

Beyond water and vegetable proteins, the Asparagus plant mainly contains amino acids, phenols, fats, steroids and saccharides as well as sodium and potassium as major trace elements. A typical characteristic of Asparagus is its saponine content. To date, many saponines have been isolated and characterised from Asparagus. They are all of the furastanol and the spirostanol types and belong to the steroidal saponines.

To date, it is still unclear, which of said components has a pharmacological effect. It is assumed, that the saponines, on the one hand, and/or the high potassium content, on the other hand, are responsible for the diuretic and, thus, the aquaretic effect.

Saponines are a group of compounds which are found in almost all diuretic medicinal plants and which are associated with their diurectic effect. However, the well-known surface activity of many of the saponines is considered to cause a number of undesirable adverse reactions encountered with the use of this group of medicinal plants and, thus, limits their field of application and the tolerable administrable dosage thereof. The latter, however, are critical factors for the therapeutic success. Particularly, the hemolytic, emetic and cytotoxic activities of the saponines limit the range of use of saponine-containing medicinal plants significantly. For example, commercial available extracts of Asparagus roots have a content of saponines in the range of 5 to 10% (based on the weight of the dried extract).

Surprisingly, it turned out that it is possible, with the process of the present invention to provide Asparagus extracts which have an excellent diuretic and anti-hypertensive effect and which simultaneously are free or almost free of undesired saponines. It was particularly surprising that, at least for derivatives of Asparagus Officinalis L. roots and/or shoots, the diuretic effect observed is not at all associated with the presence of saponines, as was repeatedly stated in the prior art. Thus, it is possible, to provide a preparation, which exhibits excellent diuretic activity but with no or almost no adverse reactions, which are typically associated with the saponines. Therefore the Asparagus extracts of the present invention are suitable for the prevention and therapy of diseases, which cannot be medicated with common Asparagus preparations, due to their adverse reaction profile.

Since many years, synthetic diuretics and among them hydrochlorothiazide (HCT) as a standard substance widely used belong—beside Ca antagonists, β blocker, ACE inhibitors and angiotensin II antagonists—to the standard therapeutica for the treatment of high blood pressure (hypertension). These diuretics are used either alone or in combination with the above-mentioned antihypertensive agents.

Because of their marked aquarese efficiency via the kidneys, diuretics have an antihypertensive and cardio protective effect.

However, the diuretics of the prior art show side effects which are undesired. In particular, hydrochlorothiazide (HCT) secrets water by the excretion of sodium and potassium which leads to a massive loss of electrolytes (shift of the electrolyte balance) in the body.

This massive shift of the electrolyte balance may lead to significant side effects, for example arrhythmia, problems of sexual potency, diabetes etc.

The present invention aims to overcome the above described disadvantages.

An object of the present invention is to provide a saponine-depleted raw extract originating from roots and/or shoots of Asparagus Officinalis L. as an intermediate which not only has diuretic and antihypertensive effects itself, but also is a broadly available starting material for saponine-free extracts from roots and/or shoots of Asparagus Officinalis L.

Another object of the present invention is to provide saponine-free extracts from said saponine-depleted raw-extract having a potent effect as diuretic and anti-hypertensive drug.

It is a further object of the present invention, to provide a method for the preparation of the saponine-depleted raw extract originating from roots and/or shoots of Asparagus Officinalis L. as an intermediate.

It is still another object of the present invention is to provide a method for the preparation of saponine-free extracts from said saponine-depleted raw-extract.

Another object of the present invention is to provide the use of said saponine-depleted raw extracts from Asparagus Officinalis L. roots and/or shoots and/or saponine-free extracts from Asparagus Officinalis L. roots and/or shoots for the promotion of a diuresis, for the promotion of purification of the body, for the promotion of body weight reduction, for the prevention and/or therapy of heart failure, for the prevention and/or therapy of hypertension and/or for the prevention and/or therapy of heart hypertrophia.

Finally, it is also an object of the present invention to provide a process for the promotion of a diuresis, for the promotion of purification of the body, for the promotion of body weight reduction, for the prevention and/or therapy of heart failure, for the prevention and/or therapy of hypertension and/or for the prevention and/or treatment of heart hypertrophia.

Hence, the invention relates to a saponine-depleted raw extract originating from roots and/or shoots of Asparagus Officinalis L. as an intermediate, obtainable by at least the steps of
grinding dry roots and/or shoots of Asparagus Officinalis L. to obtain a dry powder;
adding to the dry powder so obtained a solvent selected from the group consisting of water, at least one organic liquid miscible with water and mixtures of water and at least one organic liquid miscible with water to obtain a suspension;
agitating the suspension so obtained for a time >10 h and at a temperature not exposing the constituents of said suspension to the risk of a biological degradation;
separating the constituents of the suspension not soluble in said solvent from those constituents soluble in said solvent to obtain a soluble fraction dissolved in the solvent and an insoluble fraction; and
removing the solvent from the soluble fraction to obtain a raw extract; and optionally
grinding the raw extract obtained to prepare a saponine-depleted raw extract of roots and/or shoots of Asparagus Officinalis L. in the form of a powder.

Preferred embodiments of the saponine-depleted raw extract are claimed in subclaims 2 to 9.

The Asparagus Officinalis plant to which the present invention relates is the perennial green plant with small rhizomes to which the taxonomical classification

Class: Monocotyledonae,

Order: Liliales,

Family: Liliaceae,

Subfamily: Aparagoideae,

Genus: Asparagus,

Species: Officinalis,

applies.

As starting material for preparing the saponine-depleted raw extract of the invention, roots or shoots of Asparagus Officinalis L. are used. As used in the present specification and in the claims, the term “roots” is meant to include those parts of the Asparagus Officinalis L. plant which are remaining underground and, during cultivation, are nourishing the plant. As used in the present specification and claims, the term “shoots” is meant to include those parts of the Asparagus Officinalis L. plant which are formed by transformed leaves of the plant and, hence, are growing above the soil resulting in green shoots (due to the exposure to the natural sunlight resulting into a photosynthesis of the shoots) or are growing in raised beds shielding the shoots from the sunlight so that a white colour of the shoots prevails.

In a first step providing the source material for the extracts of the invention, the roots and/or shoots of Asparagus are reduced in size to small pieces which are carefully dried and ground to obtain a dry powder of roots and/or shoots of Asparagus Officinalis L.

In a second step, the dry powder obtained in the first step is subjected to a solvent extraction. The extraction is conducted by using one or more than one solvent. The use of one solvent is preferred. There may be used any solvent compatible with the final use, particularly the medical field use, of the extracts obtained. Useful solvents are selected from the group consisting of water, at least one organic liquid miscible with water and mixtures of water and at least one organic liquid miscible with water to obtain a suspension.

In a preferred embodiment of the invention, water is used as the solvent. Advantageously, when using water as the solvent, particularly saponine-free raw extracts are obtainable from roots and/or shoots of Asparagus Officinalis L. When practically obtaining the preferred raw extracts of roots and/or shoots of Asparagus Officinalis L. of the present invention, water is used as the solvent in an amount of from 0.1 to 10 l of water per 100 g of the dried powder of Asparagus roots and/or shoots, more preferably in an amount of from 0.5 to 5 l of water per 100 g of the dried powder of Asparagus roots and/or shoots.

Alternatively, in another preferred embodiment of the invention, at least one organic liquid miscible with water and selected from the group consisting of straight-chain or branched or cyclic alcohols having 1 to 6 carbon atoms and straight-chain or branched or cyclic ketones having 3 to 6 carbon atoms may be used as the solvent. Preferably raw extracts of Asparagus Officinalis L. roots and/or shoots are obtainable when using one or more solvent(s) selected from the group consisting of aliphatic straight-chain or branched alcohols having 1 to 6 carbon atoms, more preferably having 1 to 3 carbon atoms, and straight-chain or branched aliphatic ketones having 3 to 5 carbon atoms. Even more preferable are raw extracts from Asparagus Officinalis roots and/or shoots obtainable by using a solvent selected from methanol and ethanol and acetone and mixtures thereof. Most preferred are raw extracts obtainable by using methanol and/or ethanol and/or acetone in an amount of from 0.5 to 5 l of the solvent per 100 g of the dry powder of Asparagus Officinalis L. roots and/or shoots, utmost preferred in an amount of from 0.5 to 5 l of methanol and/or ethanol and/or acetone per 100 g of the dry powder of Asparagus Officinalis L. roots and/or shoots.

In a particularly preferred embodiment, as the solvent, a mixture of water and at least one organic liquid miscible with water and selected from the group consisting of straight-chain or branched or cyclic alcohols having 1 to 6 carbon atoms and straight-chain or branched or cyclic ketones having 3 to 6 carbon atoms may be used, preferably a mixture of water and an organic liquid selected from the group consisting of aliphatic straight-chain or branched alcohols having 1 to 6 carbon atoms, more preferably having 1 to 3 carbon atoms, and straight-chain or branched aliphatic ketones having 3 to 5 carbon atoms, even more preferable a mixture of water and an organic liquid selected from methanol and ethanol and acetone and mixtures thereof, even more preferable a mixture of water and up to 50 vol.-% of methanol and/or ethanol and/or acetone, even more preferred a mixture of water and 20 to 40 vol.-% of methanol and/or ethanol and/or acetone, even more preferred a mixture of water and 25 to 35 vol.-% of methanol and/or ethanol and/or acetone, most preferred said solvent in an amount of from 0.5 to 5 l of the solvent per 100 g of the dry powder of Asparagus Officinalis L. roots and/or shoots, utmost preferred said solvent in an amount of from 0.5 to 5 l of methanol and/or ethanol and/or acetone per 100 g of the dry powder of Asparagus Officinalis L. roots and/or shoots.

In a most preferred and particularly advantageous embodiment of the invention, the powdered Asparagus roots and/or shoots are extracted with either water or a mixture of water and 30 vol.-% ethanol so as to obtain a particularly advantageous raw extract.

In accordance with the invention, the extraction is carried out by agitating the asparagus/solvent suspension for a time >10 h and at a temperature not exposing the constituents of said suspension to the risk of a biological degradation. Particularly preferred raw extracts from Asparagus Officinalis roots and/or shoots are obtainable by shaking said suspension, more preferably said aqueous or aqueous/alcoholic suspension, for a time in the range of 10 to 48 h and/or by shaking said suspension at a temperature in the range of 10 to 25° C.

After the extraction step is completed, the constituents of the suspension not soluble in said solvent are separated from those constituents soluble in said solvent by any conceivably separation process suitable for separating liquid and solid materials. A person skilled in the art knows such separation processes and may select suitable separation steps without difficulties. Preferred raw extracts according to the invention are obtainable by means of processes selected from the group consisting of centrifugation, decantation, filtration and combinations thereof, thus obtaining a soluble fraction dissolved in the solvent and an insoluble fraction. The insoluble fraction may again be rinsed with said solvent and the soluble fractions are combined whereas the solid residue is discarded.

After the separation step, the solvent of the soluble fraction is removed. The solvent removal can be conducted by any process suitable for carefully removing solvents from solutions without taking risks of a chemical degradation of any of the components desired to be obtained. Preferred raw extracts from Asparagus Officinalis L. roots and/or shoots are obtainable either by at least one step selected from the group consisting of evaporating the solvent partly and crystallizing the solute, evaporating the solvent completely and recovering the dry solute, freeze drying the solution and combinations of those steps. As a result, a saponine-depleted raw extract of roots and/or shoots of asparagus may be obtained in good yields and having surprisingly low saponine contents.

In an optional step, the dry raw extract obtained is ground, resulting into a dry powder of an Asparagus Officinalis L. roots and/or shoots raw extract.

Furthermore, the present invention concerns a saponine-free extract from roots and/or shoots of Asparagus Officinalis L. which is obtainable from the afore described saponine-depleted raw extract by at least the steps of
adding at least one solvent selected from the group consisting of organic liquids miscible with water and mixtures of water and at least one organic liquid miscible with water, wherein the solvent has a lower hydrophilicity than the solvent used for the preparation of the raw extract, to the raw extract of any of claims 1 to 9 to obtain a suspension;
agitating the suspension so obtained for a time >30 min and at a temperature not exposing the constituents of said suspension to the risk of a biological degradation;
separating the liquid phase from any insoluble constituent to obtain a solid residue insoluble in the liquid phase; and
drying and optionally grinding the solid residue it to obtain a fractioned saponine-free extract from roots and/or shoots of Asparagus Officinalis L.

Preferred embodiments of the saponine-free extract are claimed in subclaims 11 to 20.

In the first step of preparing the saponine-free extract, at least one solvent selected from the group consisting of organic liquids miscible with water and mixtures of water and at least one organic liquid miscible with water, wherein the solvent has a lower hydrophilicity than the solvent used for the preparation of the raw extract is added as the extraction solvent to the said raw extract or to the powdered raw extract to obtain a solution or suspension.

In an alternative solution for preparing the saponine-free extract the afore-mentioned solvent for the extraction is added to a solution of the raw extract which is selected from the group consisting of water or the solvent which was used for obtaining the raw extract, to obtain a solution or suspension, wherein the solvent has a lower hydrophilicity than the solvent used for the preparation of the raw extract.

In a still further alternative, the extraction may also be carried out by adding the afore-mentioned extraction solvent directly to the solution of soluble constituents in the process for preparing the raw extract to obtain a solution or suspension, wherein the solvent has a lower hydrophilicity than the solvent used for the preparation of the raw extract. For example, the extraction may be carried out by liquid-liquid extraction (extraction in a liquid-liquid phase).

The solvent used in the above extraction-step is a solvent comprising a mixture of water and a high content of at least one organic liquid miscible with water, preferably a solvent comprising water and a high content of at least one organic liquid selected from the group consisting of straight-chain or branched or cyclic alcohols having 1 to 6 carbon atoms and straight-chain or branched or cyclic ketones having 3 to 6 carbon atoms, more preferably a solvent comprising water and a high content of an organic liquid selected from the group consisting of straight-chain or branched aliphatic alcohols having 1 to 3 carbon atoms and straight-chain or branched aliphatic ketones having 3 to 5 carbon atoms, even more preferably a solvent comprising water and a high content of methanol and/or ethanol and/or acetone, even more preferred a solvent comprising water and more than 50 vol.-% of methanol and/or ethanol and/or acetone, even more preferred a solvent comprising water and 60 to 90 vol.-% of methanol and/or ethanol and/or acetone, most preferred a solvent comprising water and 75 to 95 vol.-% of methanol and/or ethanol and/or acetone or by using, as a solvent, a purely organic liquid, preferably an organic liquid selected from the group consisting of straight-chain or branched or cyclic alcohols having 1 to 6 carbon atoms and straight-chain or branched or cyclic ketones having 3 to 6 carbon atoms, more preferably an organic liquid selected from the group consisting of straight-chain or branched aliphatic alcohols having 1 to 3 carbon atoms and straight-chain or branched aliphatic ketones having 3 to 5 carbon atoms, even more preferably an organic liquid selected from the group consisting of methanol and/or ethanol and/or acetone.

In a most preferred embodiment, the solvent for the extraction is ethanol.

For an effective extraction, the solution or suspension from the preceding step is agitated. Any agitation process may be applied for obtaining excellent saponine-free Asparagus Officinalis L. roots and/or shoots extracts. In preferred embodiments of the invention, the saponine-free extracts of the invention are obtainable for example by shaking the suspension or solution of the previous step, preferably by shaking the suspension or solution of the previous step for a time in the range of from 30 to 90 min and/or shaking the suspension or solution at a temperature in the range of from 10 to 25° C.

After the completion of the extraction-step, the liquid phase is separated from any insoluble constituent to obtain a solid residue which is insoluble in the extraction solvent of the liquid phase. Any suitable separation process may be used, without that the invention is restricted to a specific separation step. Examples of the separation step are steps of filtration, decantation, centrifugation or flotation or combinations of such separation processes.

In order to separate insoluble and soluble constituents the suspension is preferably subjected to at least one step selected from the group consisting of centrifugation, decantation, filtration and combinations of those steps so as to obtain a liquid phase and a solid residue.

Finally, the solid phase from the separation step is dried and optionally ground to obtain a fractioned saponine-free extract from roots and/or shoots of Asparagus Officinalis L.

With regard to the further described alternative embodiments, in both cases the liquid phases are separated from each other in accordance with a suitable parameter thereof and are, optionally, separated from any insoluble constituent to obtain a liquid phase with a solvent of relatively high hydrophilicity and a liquid phase with a solvent of relatively lower hydrophilicity and, optionally, a solid phase insoluble in both liquid phases.

Subsequently, the solid residue is recovered from the liquid phase with the solvent of relatively high hydrophilicity by removing the solvent, drying the obtained solid residue and optionally grinding. The product is a fractioned saponine-free extract from roots and/or shoots of Asparagus Officinalis L.

The removal of the solvent from the solution or suspension may be carried out by means of evaporating the solvent partly and crystallizing the solute, evaporating the solvent completely and recovering the dry solute, freeze drying the solution and combinations of those steps. However, the invention is not at all restricted to those steps of removing the solvent so as to obtain the solute.

The saponine-free extracts are, in particularly advantageous embodiments of the invention, prepared from an aqueous raw extract from roots or shoots of Asparagus Officinalis L or from a hydro-alcoholic, preferably from a hydroethanolic, more preferably from a hydro-(20- to 40-vol.-% ethonolic) raw extract from roots or shoots of Asparagus Officinalis L.

In a preferred embodiment of the present invention, the saponine-free extract is prepared using an aqueous raw extract from roots or shoots of Asparagus Officinalis L.

The raw extracts from roots and/or shoots of Asparagus Officinalis L. have a saponine content of <5% by weight, preferably of 2 to 3% by weight, even more preferred of <2% by weight.

The fractioned saponine-free extracts from roots and/or shoots of Asparagus Officinalis L. are substantially saponine-free.

In addition, the present invention concerns a process for preparing a saponine-depleted raw extract from roots and/or shoots of Asparagus Officinalis, comprising the steps of
grinding dry roots and/or shoots of Asparagus Officinalis L. to obtain a dry powder;
adding to the dry powder so obtained a solvent selected from the group consisting of water, at least one organic liquid miscible with water and mixtures of water and at least one organic liquid miscible with water to obtain a suspension;
agitating the suspension so obtained for a time >10 h and at a temperature not exposing the constituents of said suspension to the risk of a biological degradation;
separating the constituents of the suspension not soluble in said solvent from those constituents soluble in said solvent to obtain a soluble fraction dissolved in the solvent and an insoluble fraction; and
removing the solvent from the soluble fraction to obtain a raw extract; and optionally
grinding the raw extract obtained to prepare a saponine-depleted raw extract of roots and/or shoots of Asparagus Officinalis L. in the form of a powder.

Preferred embodiments of said process are claimed in subclaims 24 to 31.

As starting material for preparing the saponine-depleted raw extract roots or shoots of Asparagus Officinalis L. are used.

In a first step of the process the roots and/or shoots of Asparagus are reduced to small pieces, dried and ground to obtain a dry powder.

In a second step, the powder is extracted by using different solvents, which are selected from the group consisting of water, at least one organic liquid miscible with water and mixtures of water and at least one organic liquid miscible with water to obtain a suspension. In a preferred embodiment water is used as the solvent in an amount of from 0.1 to 10 l of water per 100 g of the dried powder of asparagus roots and/or shoots, more preferably in an amount of from 0.5 to 5 l of water per 100 g of the dried powder of asparagus roots and/or shoots.

Alternatively, as the solvent, at least one organic liquid miscible with water and selected from the group consisting of straight-chain or branched or cyclic alcohols having 1 to 6 carbon atoms and straight-chain or branched or cyclic ketones having 3 to 6 carbon atoms, preferably selected from the group consisting of aliphatic straight-chain or branched alcohols having 1 to 6 carbon atoms, more preferably having 1 to 3 carbon atoms, and straight-chain or branched aliphatic ketones having 3 to 5 carbon atoms, even more preferable selected from methanol and ethanol and acetone and mixtures thereof, most preferred in an amount of from 0.5 to 5 l of the solvent per 100 g of the dry powder of Asparagus Officinalis L. roots and/or shoots, utmost preferred in an amount of from 0.5 to 5 l of methanol and/or ethanol and/or acetone per 100 g of the dry powder of Asparagus Officinalis L. roots and/or shoots may be used.

In a preferred embodiment, as the solvent, a mixture of water and at least one organic liquid miscible with water and selected from the group consisting of straight-chain or branched or cyclic alcohols having 1 to 6 carbon atoms and straight-chain or branched or cyclic ketones having 3 to 6 carbon atoms, preferably a mixture of water and an organic liquid selected from the group consisting of aliphatic straight-chain or branched alcohols having 1 to 6 carbon atoms, more preferably having 1 to 3 carbon atoms, and straight-chain or branched aliphatic ketones having 3 to 5 carbon atoms, even more preferable a mixture of water and an organic liquid selected from methanol and ethanol and acetone and mixtures thereof, even more preferable a mixture of water and up to 50 vol.-% of methanol and/or ethanol and/or acetone, even more preferred a mixture of water and 20 to 40 vol.-% of methanol and/or ethanol and/or acetone, even more preferred a mixture of water and 25 to 35 vol.-% of methanol and/or ethanol and/or acetone, most preferred said solvent in an amount of from 0.5 to 5 l of the solvent per 100 g of the dry powder of Asparagus Officinalis L. roots and/or shoots, utmost preferred said solvent in an amount of from 0.5 to 5 l of methanol and/or ethanol and/or acetone per 100 g of the dry powder of Asparagus Officinalis L. roots and/or shoots may be used.

In a most preferred embodiment, the powdered Asparagus roots and/or shoots are extracted with either water or a mixture of water and 30 vol.-% ethanol.

The extraction is carried out by agitating the asparagus/solvent suspension for a time >10 h and at a temperature not exposing the constituents of said suspension to the risk of a biological degradation, preferably by shaking said suspension for a time in the range of 10 to 48 h and/or shaking said suspension at a temperature in the range of 10 to 25° C.

After the extraction step is completed, the constituents of the suspension not soluble in said solvent are separated from those constituents soluble in said solvent by means of centrifugation, decantation, filtration and combinations thereof and thus obtaining a soluble fraction dissolved in the solvent and an insoluble fraction. The insoluble faction is again rinsed with said solvent and the solvent fractions are combined whereas the solid residue is discarded.

After the separation step, the solvent of the soluble fraction is removed either by at least one step selected from the group consisting of evaporating the solvent partly and crystallizing the solute, evaporating the solvent completely and recovering the dry solute, freeze drying the solution and combinations of those steps to prepare a saponine-depleted raw extract of roots and/or shoots of asparagus.

In an optionally step the dry raw extract obtained is ground.

Furthermore, the present invention concerns a process for preparing a saponine-free extract from roots and/or shoots of Asparagus Officinalis L. which is obtainable from the afore described saponine-depleted raw extract by at least the steps of
adding at least one solvent selected from the group consisting of organic liquids miscible with water and mixtures of water and at least one organic liquid miscible with water, wherein the solvent has a lower hydrophilicity than the solvent used for the preparation of the raw extract, to the raw extract of any of claims 1 to 9 to obtain a suspension;
agitating the suspension so obtained for a time >30 min and at a temperature not exposing the constituents of said suspension to the risk of a biological degradation;
separating the liquid phase from any insoluble constituent to obtain a solid residue insoluble in the liquid phase; and
drying and optionally grinding the solid residue it to obtain a fractioned saponine-free extract from roots and/or shoots of Asparagus Officinalis L.

Preferred embodiments of the process for preparing a saponine-free extract are claimed in subclaims 32 to 42.

In the first step of preparing the saponine-free extract, at least one solvent selected from the group consisting of organic liquids miscible with water and mixtures of water and at least one organic liquid miscible with water, wherein the solvent has a lower hydrophilicity than the solvent used for the preparation of the raw extract is added to the raw extract or to the powdered raw extract to obtain a solution or suspension.

In an alternative solution for preparing the saponine-free extract the afore-going solvent for the extraction is added to a solution of the raw extract which is selected from the group consisting of water or the solvent which was used for obtaining the raw extract, to obtain a solution or suspension.

In a still further alternative the extraction may also be carried out by adding the afore-going solvent directly to the solution of soluble constituents in the process for preparing the raw extract to obtain a solution or suspension. For example, the extraction may be carried out by liquid-liquid extraction (extraction on a liquid-liquid phase).

The solvent used in the above extraction-step is a solvent comprising a mixture of water and a high content of at least one organic liquid miscible with water, preferably a solvent comprising water and a high content of at least one organic liquid selected from the group consisting of straight-chain or branched or cyclic alcohols having 1 to 6 carbon atoms and straight-chain or branched or cyclic ketones having 3 to 6 carbon atoms, more preferably a solvent comprising water and a high content of an organic liquid selected from the group consisting of straight-chain or branched aliphatic alcohols having 1 to 3 carbon atoms and straight-chain or branched aliphatic ketones having 3 to 5 carbon atoms, even more preferably a solvent comprising water and a high content of methanol and/or ethanol and/or acetone, even more preferred a solvent comprising water and more than 50 vol.-% of methanol and/or ethanol and/or acetone, even more preferred a solvent comprising water and 60 to 90 vol.-% of methanol and/or ethanol and/or acetone, most preferred a solvent comprising water and 75 to 95 vol.-% of methanol and/or ethanol and/or acetone or by using, as a solvent, a purely organic liquid, preferably an organic liquid selected from the group consisting of straight-chain or branched or cyclic alcohols having 1 to 6 carbon atoms and straight-chain or branched or cyclic ketones having 3 to 6 carbon atoms, more preferably an organic liquid selected from the group consisting of straight-chain or branched aliphatic alcohols having 1 to 3 carbon atoms and straight-chain or branched aliphatic ketones having 3 to =5 carbon atoms, even more preferably an organic liquid selected from the group consisting of methanol and/or ethanol and/or acetone.

In a most preferred embodiment, the solvent for the extraction is ethanol.

For an effective extraction, the solution or suspension from the preceding step is agitated, for example by shaking the suspension or solution, preferably by shaking the suspension or solution for a time in the range of from 30 to 90 min and/or shaking the suspension or solution at a temperature in the range of from 10 to 25° C.

After the completion of the extraction-step, the liquid phase is separated from any insoluble constituent to obtain a solid residue which is insoluble in the liquid phase. In order to separate insoluble and soluble constituents the suspension is subjected at least one step selected from the group consisting of centrifugation, decantation, filtration and combinations of those steps obtaining a liquid phase and a solid residue.

Finally, the solid phase from the separation step is dried and optionally ground to obtain a fractioned saponine-free extract from roots and/or shoots of Asparagus Officinalis L.

With regard to the further described alternative embodiments in both cases the liquid phases are separated in accordance with a suitable parameter thereof from each other and, optionally, from any insoluble constituent to obtain a liquid phase with a solvent of relatively high hydrophilicity and a liquid phase with a solvent of relatively lower hydrophilicity and, optionally a solid phase insoluble in both liquid phases.

Subsequently, the solid residue is recovered from the liquid phase with the solvent of relatively high hydrophilicity by removing the solvent, drying the obtained solid residue and optionally grinding. The product is a fractioned saponine-free extract from roots and/or shoots of Asparagus Officinalis L.

The removal of the solvent from the solution or suspension may be carried out by means of evaporating the solvent partly and crystallizing the solute, evaporating the solvent completely and recovering the dry solute, freeze drying the solution and combinations of those steps.

The saponine-free extracts are prepared from an aqueous raw extract from roots or shoots of Asparagus Officinalis L or from a hydro-alcoholic, preferably from a hydroethanolic, more preferably from a hydro-(20- to 40-vol.-% ethanolic) raw extract from roots or shoots of Asparagus Officinalis L.

In a preferred embodiment of the present invention, the saponine-free extract is prepared using an aqueous raw extract from roots or shoots of Asparagus Officinalis L.

Furthermore, the present invention relates to a pharmaceutical preparation or nutritional supplement, in particular a vegetable diuretic preparation comprising one or more of the components of saponine-depleted raw extracts from Asparagus Officinalis L. roots and/or shoots and/or one or more of saponine-free extracts from Asparagus Officinalis L. roots and/or shoots as defined above.

These preparations may optionally comprise one or more pharmacologically acceptable carrier substances, auxiliary substances and/or vehicles. Furthermore, the preparations may further comprise vegetable extracts which are selected from broccoli, sweet corn, stining-nettle, parsley, pineapple, pawpaw, mango etc. which have an additive diuretic effect. In addition, vitamins like vitamin A, vitamin B, vitamin C, vitamin E, biotin, mineral nutrients, trace elements and/or natural flavouring substances may be added to the preparation.

In a most preferred embodiment, the pharmaceutical preparation or nutritional supplement, in particular vegetable diuretic preparation comprises the saponine-free ethanol-insoluble component of the aqueous or of the hydro-alcoholic raw extract of Asparagus Officinalis L. roots and/or shoots.

Moreover, the present invention relates to the use of one or more of the components of saponine-depleted raw extracts from Asparagus Officinalis L. roots and/or shoots and/or one or more of saponine-free extracts from Asparagus Officinalis L. roots and/or shoots as defined above for the manufacture of a medicament for the promotion of a diuresis, for the promotion of purification of the body, for the promotion of body weight reduction, for the prevention and/or treatment of heart failure, for the prevention and/or treatment of hypertension and/or for the prevention and/or treatment of heart hypertrophia.

In addition, the present invention relates to a process for the promotion of a diuresis, for the promotion of purification of the body, for the promotion of body weight reduction, for the prevention and/or treatment of heart failure, for the prevention and/or treatment of hypertension and/or for the prevention and/or treatment of heart hypertrophia in a mammal, preferably in a human, comprising an administration of one or more of the components of saponine-depleted raw extracts from Asparagus Officinalis L. roots and/or shoots and/or one or more of saponine-free extracts from Asparagus Officinalis L. roots and/or shoots as described in detail above to a mammal, preferably to a human, in need of such a promotion of conditions and/or prevention and/or treatment of diseases in an amount affording an effective promotion of conditions and/or prevention and/or treatment of diseases.

### BRIEF DESCRIPTION OF THE DRAWINGS

FIG. 1 is a flow sheet showing the steps for the preparation of the saponine-depleted raw extract.

FIG. 2 is a flow sheet showing the steps for the extraction for preparing the saponine-free fractioned extract.

FIG. 3 is a diagram showing the cumulative mean volume of urine depending on the time for extracts “roots”.

FIG. 4 is a diagram showing the cumulative mean volume of urine depending on the time for extracts “shoots”.

FIG. 5 is diagram showing the cumulative mean volume of urine excreted by rats depending on the time for fractioned aqueous extracts of “shoots”.

The present invention will now be described in more detail by the following Examples. It is intended that the Examples explain, but do not restrict the invention.

### EXAMPLE 1

### Preparation of Different Extracts of Asparagus Officinalis L.

Vegetable Extracts

The extracts used were prepared form roots and shoots of Asparagus Officinalis L., independently.

Extracts Prepared from Parts of Roots:

aqueous raw extract:

### PREPARATION EXAMPLE 1

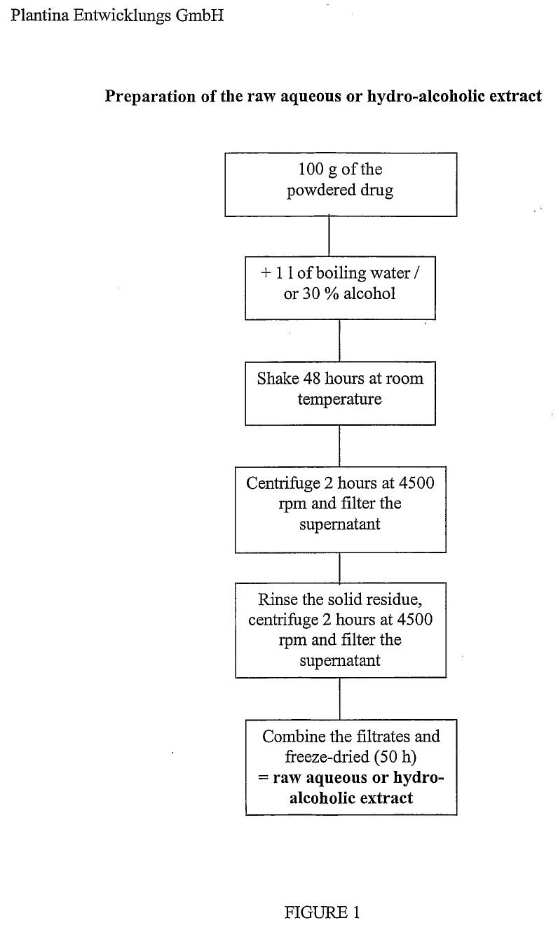

100 g of the Asparagus Officinalis L. roots in the form of a powder dried carefully under controlled conditions were suspended in 1.000 ml of water. The suspension was shaken at 20° C. (room temperature) for 48 h. The thus obtained mixed suspension was subjected to centrifugation at 4.500 rpm for 2 hours. The solid residue was rinsed with water and again subjected to a centrifugation step at 4.500 rpm for 2 hours. The solid residue resulting from said second centrifugation step was discarded. The supernatants of the two centrifugation steps were combined, and the water was removed by freeze-drying. There was obtained a dark-brown solid in a yield of 11% by weight, based on the weight of the starting roots powder. The sodium content was 125 mg and the potassium content was 121 mg (based on 100 g of dried plant starting material). This process is shown in the scheme of FIG. 1.
hydro-alcoholic raw extract:

### PREPARATION EXAMPLE 2

The above procedure (of obtaining the aqueous raw extract; preparation example 1) was repeated, with the exception that a mixture of 30 vol.-% ethanol and 70 vol.-% water was used for the extraction. The product was a light-brown solid in a yield of 10% by weight of the starting roots powder. The sodium content was 126 mg and the potassium content was 106 mg (based on 100 g of dried plant starting material). This process is shown in the scheme of FIG. 1.
fractioned aqueous extract:

### PREPARATION EXAMPLE 3

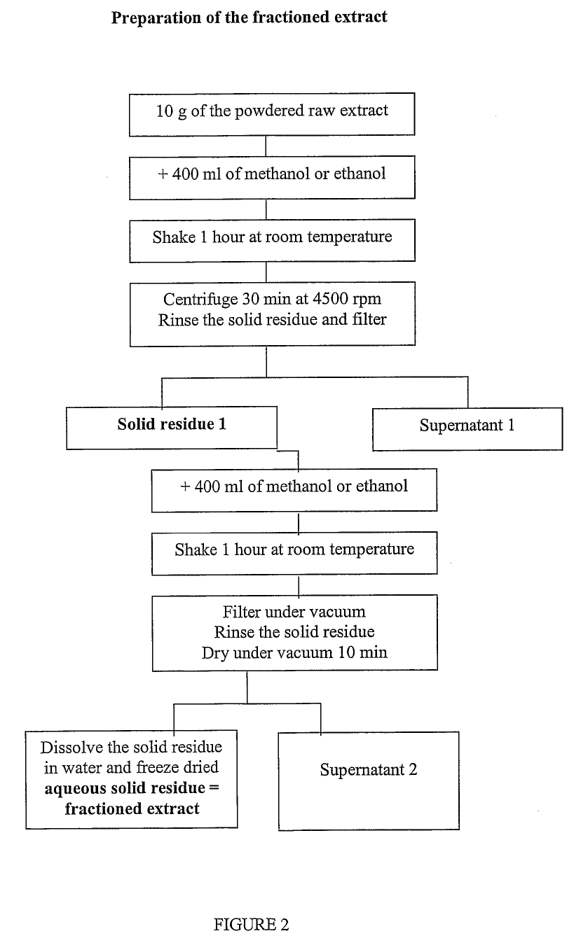

The dry powder of the aqueous raw extract of preparation example 1 (10 g) was suspended in 400 ml ethanol. The suspension was shaken for 60 min at 20° C. (ambient temperature). The suspension thus obtained was subjected to centrifugation at 4.500 rpm for 30 minutes. The solid residue was rinsed with 400 ml ethanol, subjected to filtration and dried under vacuum. The solid residue resulting from said drying step was recovered; yield: 56% by weight, based on the amount of raw extract employed. The sodium content was 90 mg and the potassium content was 78 mg (based on 100 g of dried plant starting material). The contents of the supernatant of the centrifugation and filtration steps were not further considered for the further study. This process is shown in the scheme of FIG. 2.
fractioned hydro-alcoholic extract:

### PREPARATION EXAMPLE 4

The dry powder of the hydro-alcoholic raw extract of preparation example 2 (10 g) was suspended in 400 ml ethanol. The suspension was shaken for 60 min at 20° C. (ambient temperature). The suspension thus obtained was subjected to centrifugation at 4.500 rpm for 30 minutes. The solid residue was rinsed with 400 ml ethanol, subjected to filtration and dried under vacuum. The solid residue resulting from said drying step was recovered; yield: 47% by weight, based on the amount of raw extract employed. The sodium content was 35 mg and the potassium content was 19 mg (based on 100 g of dried plant starting material). The contents of the supernatant of the centrifugation and filtration steps were not further considered for the further study. This process is shown in the scheme of FIG. 2.

Extracts Prepared from Parts of Shoots:

aqueous raw extract:

### PREPARATION EXAMPLE 5

100 g of the Asparagus Officinalis L. shoots in the form of a powder dried carefully under controlled conditions were suspended in 1.000 ml of water. The suspension was shaken at 20° C. (room temperature) for 48 h. The thus obtained mixed suspension was subjected to centrifugation at 4.500 rpm for 2 hours. The solid residue was rinsed with water and again subjected to a centrifugation step at 4.500 rpm for 2 hours. The solid residue resulting from said second centrifugation step was discarded. The supernatants of the two centrifugation steps were combined, and the water was removed by freeze-drying. There was obtained a light-brown solid in a yield of 31% by weight, based on the weight of the starting roots powder. The sodium content was 198 mg and the potassium content was 1,954 mg (based on 100 g of dried plant starting material). This process is shown in the scheme of FIG. 1.
hydro-alcoholic raw extract:

### PREPARATION EXAMPLE 6

The above procedure (of obtaining the aqueous raw extract; preparation example 5) was repeated, with the exception that a mixture of 30 vol.-% ethanol and 70 vol.-% water was used for the extraction. The product was a light-brown solid in a yield of 33% by weight of the starting roots powder. The sodium content was 210 mg and the potassium content was 2,210 mg (based on 100 g of dried plant starting material). This process is shown in the scheme of FIG. 1.
fractioned aqueous extract:

### PREPARATION EXAMPLE 7

The dry powder of the aqueous raw extract of preparation example 5 (10 g) was suspended in 400 ml ethanol. The suspension was shaken for 60 min at 20° C. (ambient temperature). The suspension thus obtained was subjected to centrifugation at 4.500 rpm for 30 minutes. The solid residue was rinsed with 400 ml ethanol subjected to filtration and dried under vacuum. The solid residue resulting from said drying step was recovered. The contents of the supernatant of the centrifugation and filtration steps were not further considered for the further study. This process is shown in the scheme of FIG. 2.
fractioned hydro-alcoholic extract:

### PREPARATION EXAMPLE 8

The dry powder of the hydro-alcoholic raw extract of preparation example 6 (10 g) was suspended in 400 ml ethanol. The suspension was shaken for 60 min at 20° C. (ambient temperature). The suspension thus obtained was subjected to centrifugation at 4.500 rpm for 30 minutes. The solid residue was rinsed with ethanol, subjected to filtration and dried under vacuum. The solid residue resulting from said drying step was recovered. The contents of the supernatant of the centrifugation and filtration steps were not further considered for the further study. This process is shown in the scheme of FIG. 2.

The saponine contents of the starting materials (powders of roots or shoots of Asparagus Officinalis L.), raw extracts and fractioned extracts of roots or shoots of Asparagus Officinalis L. were determined by thin layer chromatography on silica gel. The results revealed a relatively high saponine content of the starting materials (as expected), while in the raw extracts, the saponine content was considerably reduced. In the solid residues of the ethanolic fractionation steps (preparation examples 3, 4, 7 and 8), the thin layer chromatography showed substantially saponine-free products.

### EXAMPLE 2

### Diuretic and Antihypertensive Effects of Different Extracts of Asparagus Officinalis L.

Animals

The animals employed in the tests were male OFA Sprague-Dawley rats (obtained from the Centre d'elevage Iffa-Credo), each having a weight in the range of from 225 to 250 g. All the animals later employed in the tests were held in individual cages at a temperature of 21±1° C. and were subjected to a day/night cycle of 12/12 h for 8 days to adapt them to the test environment. During this phase, the animals were fed a standardized feed (croquettes M 20; SDS, G.B.) and water ad libitum.

Experimental Protocol

Adaptation

48 h before the start of the experiment the rats were placed in a cage allowing separate feeding and individual metabolism of each of the animals. Feed and drink (water) ad libitum.

Experimentation

After the above 48 h period, each animal was administered an amount of 5 ml of an aqueous NaCl solution (0.45% NaCl) per 100 g body weight of the animal on the intraperitoneal administration route, to which solution was added the product to be tested in the case of the treated animal group. In the cases of animals of the control group, only the saline solution was administered intraperitoneally. The animals were held under diet for 8 h after the administration in their metabolism cages. The urine of all animals was collected in graduated polypropylene vials, and their volumes were noted every hour during the 8 h period. After 8 h, the vials were removed from the animals, the urine pH value was measured (portable pH meter: PHM 201 MeterlabR; Choffel, France), and the contents of K⁺ and Na⁺ were measured by emission spectrometry (Spectraspan VII; Spectrametrics).

Tests
Control group: NaCl 0.45% (n=10 rats)
Reference group: hydrochlorothiazide (EsidrixR; Ciba-Geigy) 10 mg/kg weight (n=10 rats); hydrochlorothiazide is used as standard diuretic.
Vegetable extract “roots” group: each extract was tested on the basis of 400 mg starting material (dried plant material)/kg weight; The number of animals per group was 10 for the aqueous (preparation example 1) and hydro-alcoholic (preparation example 2) raw extracts and 12 animals each for the fractioned aqueous (preparation example 3) and fractioned hydro-alcoholic (preparation example 4) extracts;
Vegetable extraxt “shoots” group: each raw extract was tested in two different concentrations, namely 400 mg and 116 mg dried plant/kg weight for the aqueous raw extract (preparation example 5) and 400 mg and 109 mg dried plant/kg weight for the hydro-alcoholic raw extract (preparation example 6);
The number of animals per group was 10 in each case.
   (4) Diuretic Effects:

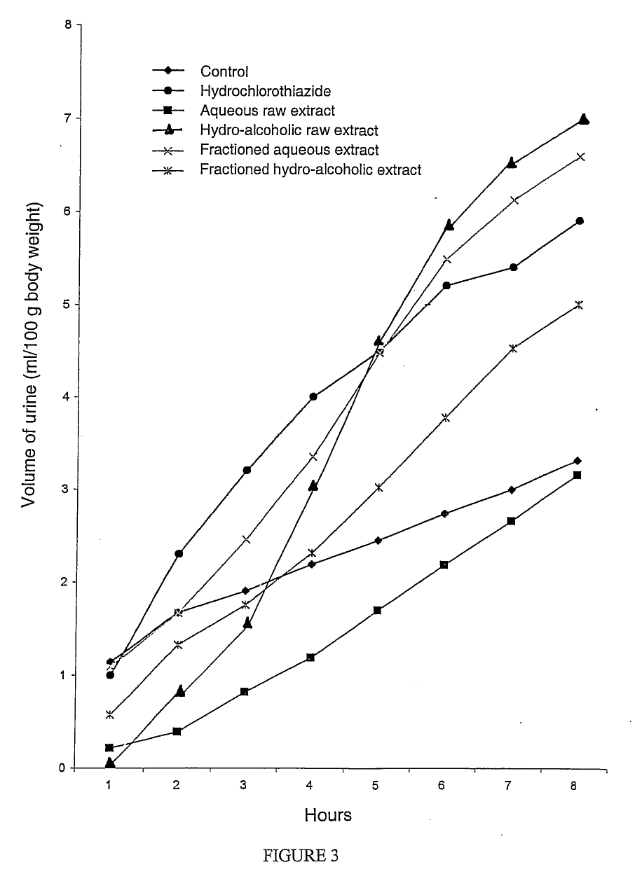

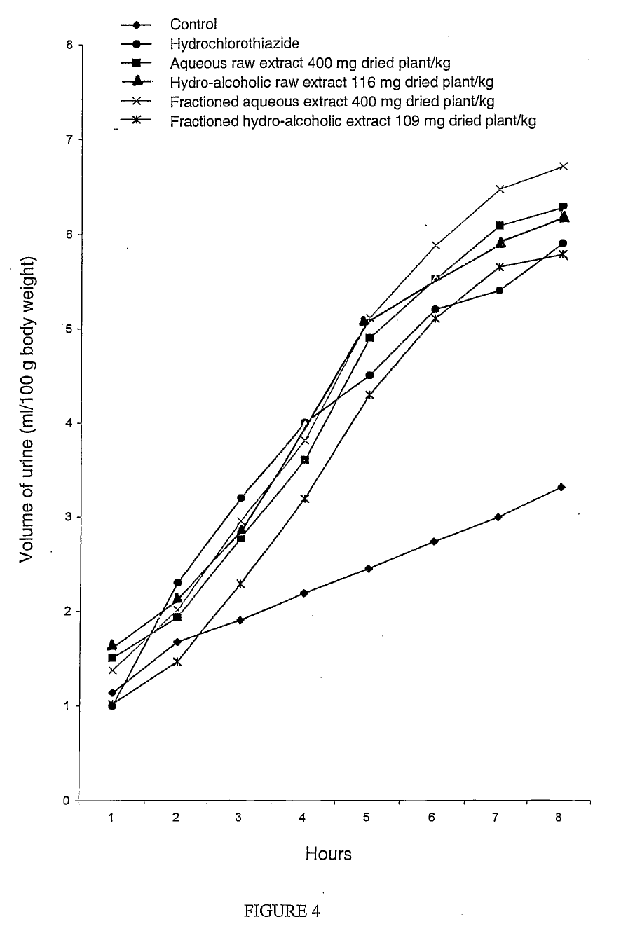

The diuretic effect of the extracts of preparation examples 1 to 4 and of the extracts of preparation examples 5 and 6 are shown in FIGS. 3 and 4, respectively.

It is obvious from FIG. 3 that, with respect to extracts originating from Asparagus roots, the hydro-alcoholic raw extract (product of preparation example 2), the fractioned aqueous extract (product of preparation example 3), and the fractioned hydro-alcoholic extract (product of preparation example 4) had excellent diuretic effects comparable to or even better than hydrochlorothiazide which is a potent diuretic of the prior art, if administered in the above concentrations.

As shown in FIG. 4, with respect to extracts originating from Asparagus shoots, the aqueous raw extract (product of preparation example 5) and the hydro-alcoholic raw extract (product of preparation example 6) in an administration dose of 400 mg dried plant/kg body weight showed considerably better diuretic effects than the reference substance hydrochlorothiazide (standard diuretic). The hydro-alcoholic raw extract (product of preparation example 6) if administered in an amount of 109 mg dried plant/kg body weight showed a diuretic effect similar to the effect exerted by the reference substance hydrochlorothiazide.

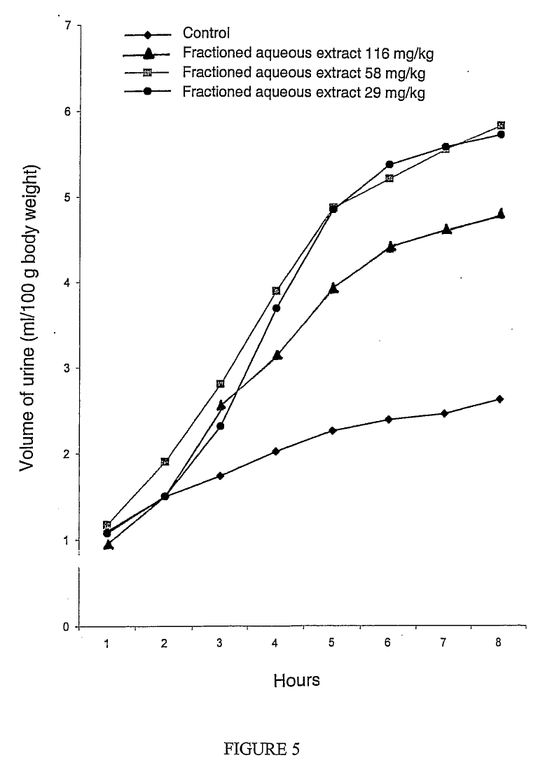

A surprising result was found with respect to the fractioned aqueous extracts of Asparagus shoots (preparation example 7): If administered in (a) an amount of 116 mg (basis: dried plant starting material) per kg body weight; (b) an amount of 58 mg (basis: dried plant starting material) per kg body weight; and (c) an amount of 29 mg (basis: dried plant starting material) per kg body weight, the latter two administrations exert considerably better results than the former one (a). This is shown in FIG. 5. In other words: Lower concentrations of the diuretically effective extracts, administered under comparable conditions, result into better effects than the higher concentration of the vegetable diuretic.

(5) Evaluation of the Results:

To summarise the above results it can be stated that the extracts of the present invention exhibit excellent diuretic effects compared to the standard diuretic substance hydrochlorothiazide (HCT).

Furthermore, the diuretic effect—as known in the art—is accompanied by an antihypertensive effect not only in combination with other hypertensive drugs but also when administered alone.

With the extracts of the present invention the disadvantageous side effects caused by the simultaneously excretion of sodium and potassium ions can be diminished: The extracts of the present invention contain sodium and potassium in considerable amounts. Due to the alkali metal ion content (see preparation examples) the administration of the inventive extracts compensates the diuretic excretion of sodium and potassium ions widely. The improvement of the electrolyte balance overcomes the disadvantages of the prior art diuretics.

1.-49. (canceled)

50. A substantially saponine-free raw extract from at least one of roots and shoots of Asparagus Officinalis L., obtainable by a process comprising
(a) combining at least one of water and a water-miscible organic liquid with a dry powder obtainable by grinding at least one of dry roots and dry shoots of Asparagus Officinalis L. to obtain a suspension;
(b) agitating the suspension of (a) for at least 10 hours at a temperature which does not result in a substantial degradation of biological material comprised in the suspension;
(c) separating undissolved matter in the suspension from a liquid phase to obtain a soluble fraction dissolved in the liquid phase and an insoluble fraction;
(d) removing liquid from the soluble fraction of (c) to obtain a raw extract;
(e) optionally, grinding the raw extract of (d) to provide a raw extract of at least one of roots and shoots of Asparagus Officinalis L. in powder form;
(1) combining the soluble fraction dissolved in the liquid phase of (c) with a solvent selected from one or more water-miscible organic liquids and mixtures of one or more water-miscible organic liquids and water, wherein the solvent has a lower hydrophilicity than the liquid used for the preparation of the raw extract to obtain a solution or suspension; or
(1′) combining a solution of the raw extract obtained in at least one of (d) and (e) in a solvent selected from water and the water-miscible organic liquid used for the preparation of the raw extract with a solvent selected from one or more water-miscible organic liquids and mixtures of one or more water-miscible organic liquids and water, wherein the solvent has a lower hydrophilcity than the liquid used for the preparation of the raw extract to obtain a solution or suspension; or
(1″) combining the raw extract obtained in at least one of (d) and (e) with a solvent selected from one or more water-miscible organic liquids and mixtures of one or more water-miscible organic liquids and water, wherein the solvent has a lower hydrophilicity than the liquid used for the preparation of the raw extract to obtain a suspension;
(2) agitating the solution or suspension obtained in (1), (1′) or (1″) for at least 30 min and at a temperature which does not result in a substantial degradation of biological material comprised in the solution or suspension;
(3) in the case of (1) or (1′), separating liquid phases from each other in accordance with a suitable parameter thereof and, optionally, from any insoluble constituent to obtain a liquid phase with a solvent of relatively high hydrophilicity and a liquid phase with a solvent of relatively lower hydrophilicity and, optionally, a solid phase which is insoluble in both liquid phases or,
(3′) in the case of (1″), separating the liquid phase from any insoluble constituent to obtain a solid residue which is insoluble in the liquid phase;
(4) in the case of (3), removing, from the liquid phase with the solvent of relatively high hydrophilicity, the liquid to obtain a solid residue; and
(5) drying and optionally grinding the solid residue to obtain a fractioned substantially saponine-free extract from at least one of roots and shoots of Asparagus Officinalis L.

51. The extract of claim 50, wherein in (a) water is used as solvent.

52. The extract of claim 51, wherein in (a) from 0.1 to 10 L of water per 100 g of a dry powder is used.

53. The extract of claim 50, wherein in (a) at least one water-miscible liquid selected from straight-chain, branched and cyclic alcohols having from 1 to 6 carbon atoms and straight-chain, branched and cyclic ketones having from 3 to 6 carbon atoms is used as solvent.

54. The extract of claim 53, wherein in (a) at least one water-miscible liquid selected from methanol, ethanol and acetone is used.

55. The extract of claim 54, wherein in (a) from 0.5 to 5 L of one or more of methanol, ethanol and acetone is used per 100 g of dry powder.

56. The extract of claim 50, wherein in (a) a mixture of water and at least one water-miscible liquid selected from straight-chain, branched and cyclic alcohols having from 1 to 6 carbon atoms and straight-chain, branched and cyclic ketones having from 3 to 6 carbon atoms is used as solvent.

57. The extract of claim 56, wherein the at least one water-miscible liquid is selected from one or more of methanol, ethanol and acetone.

58. The extract of claim 57, wherein the mixture comprises up to 50 vol.-% of one or more of methanol, ethanol and acetone.

59. The extract of claim 56, wherein in (a) from 0.5 to 5 L of the solvent is used per 100 g of dry powder of (a).

60. The extract of claim 50, wherein (b) comprises shaking the suspension for from 10 to 48 h.

61. The extract of claim 50, wherein (b) comprises shaking the suspension at a temperature of from 10° C. to 25° C.

62. The extract of claim 50, wherein (c) comprises at least one operation selected from centrifugation, decantation and filtration.

63. The extract of claim 50, wherein (d) comprises at least one of (i) partially evaporating the liquid and crystallizing a solute, (ii) completely evaporating the liquid and recovering a dry solute, and (iii) freeze drying the soluble fraction.

64. The extract of claim 50, wherein in (a) a powder derived from roots of Asparagus Officinalis L. is employed.

65. The extract of claim 50, wherein in (a) a powder derived from shoots of Asparagus Officinalis L. is employed.

66. The extract of claim 50, wherein the solvent used in (1), (1′) or (1″) comprises more than 50 vol.-% of at least one water-miscible organic liquid selected from straight-chain, branched and cyclic alcohols having from 1 to 6 carbon atoms and straight-chain, branched and cyclic ketones having from 3 to 6 carbon atoms.

67. The extract of claim 50, wherein the solvent used in (1), (1′) or (1″) comprises more than 50 vol.-% of at least one of methanol, ethanol and acetone.

68. The extract of claim 67, wherein the solvent used in (1), (1′) or (1″) comprises from 75 to 95 vol.-% of at least one of methanol, ethanol and acetone.

69. The extract of claim 50, wherein the solvent used in (1), (1′) or (1″) consists essentially of one or more water-miscible organic liquids selected from straight-chain, branched and cyclic alcohols having from 1 to 6 carbon atoms and straight-chain, branched and cyclic ketones having from 3 to 6 carbon atoms.

70. The extract of claim 69, wherein the solvent used in (1), (1′) or (1″) consists essentially of one or more of methanol, ethanol and acetone.

71. The extract of claim 50, wherein (2) comprises shaking the suspension or solution for from 30 to 90 min.

72. The extract of claim 50, wherein (2) comprises shaking the suspension or solution at a temperature of from 10° C. to 25° C.

73. The extract of claim 50, wherein (3) or (3′) comprises at least one operation selected from centrifugation, decantation and filtration.

74. The extract of claim 50, wherein (4) comprises at least one of (i) partially evaporating the liquid and crystallizing a solute, (ii) completely evaporating the liquid and recovering a dry solute, and (iii) freeze drying the liquid phase.

75. The extract of claim 50, wherein the raw extract comprises an aqueous raw extract of roots of Asparagus Officinalis L.

76. The extract of claim 50, wherein the raw extract comprises an aqueous raw extract of shoots of Asparagus Officinalis L.

77. The extract of claim 50, wherein the raw extract comprises a hydroalcoholic raw extract of roots of Asparagus Officinalis L.

78. The extract of claim 50, wherein the raw extract comprises a hydroalcoholic raw extract of shoots of Asparagus Officinalis L.

79. A process for preparing a saponine-depleted raw extract from at least one of roots and shoots of Asparagus Officinalis L., which process comprises
(a) combining at least one of water and a water-miscible organic liquid with a dry powder obtainable by grinding at least one of dry roots and dry shoots of Asparagus Officinalis L. to obtain a suspension;
(b) agitating the suspension of (a) for at least 10 hours at a temperature which does not result in a substantial degradation of biological material comprised in the suspension;
(c) separating undissolved matter in the suspension from a liquid phase to obtain a soluble fraction dissolved in the liquid phase and an insoluble fraction;
(d) removing liquid from the soluble fraction of (c) to obtain a raw extract; and
(e) optionally, grinding the raw extract of (d) to provide the extract in powder form.

80. The process of claim 79, wherein the saponine-depleted raw extract has a saponine content of less than 5% by weight.

81. A process for preparing a substantially saponine-free extract of Asparagus Officinalis L. from the saponine-depleted raw extract obtainable by the process of claim 79, which process comprises
(1) combining the soluble fraction dissolved in the liquid phase of (c) with a solvent selected from one or more water-miscible organic liquids and mixtures of one or more water-miscible organic liquids and water, wherein the solvent has a lower hydrophilicity than the liquid used for the preparation of the raw extract to obtain a solution or suspension; or
(1′) combining a solution of the raw extract obtained in at least one of (d) and (e) in a solvent selected from water and the water-miscible organic liquid used for the preparation of the raw extract with a solvent selected from one or more water-miscible organic liquids and mixtures of one or more water-miscible organic liquids and water, wherein the solvent has a lower hydrophilicity than the liquid used for the preparation of the raw extract to obtain a solution or suspension; or
(1″) combining the raw extract obtained in at least one of (d) and (e) with a solvent selected from one or more water-miscible organic liquids and mixtures of one or more water-miscible organic liquids and water, wherein the solvent has a lower hydrophilicity than the liquid used for the preparation of the raw extract to obtain a suspension;
(2) agitating the solution or suspension of (1), (1′) or (1″) for at least 30 min and at a temperature which does not result in a substantial degradation of biological material comprised in the suspension;
(3) in the case of (1) or (1′), separating liquid phases from each other in accordance with a suitable parameter thereof and, optionally, from any insoluble constituent to obtain a liquid phase with a solvent of relatively high hydrophilicity and a liquid phase with a solvent of relatively lower hydrophilicity and, optionally, a solid phase which is insoluble in both liquid phases or,
(3′) in the case of (1″), separating the liquid phase from any insoluble constituent to obtain a solid residue which is insoluble in the liquid phase;
(4) in the case of (3), removing, from the liquid phase with the solvent of relatively high hydrophilicity, the liquid to obtain a solid residue; or
(5) drying and optionally grinding the solid residue to obtain a fractioned substantially saponine-free extract from at least one of roots and shoots of Asparagus Officinalis L.

82. A pharmaceutical preparation or nutritional supplement, wherein the preparation or supplement comprises one or more extracts according to claim 50, optionally in combination with one or more pharmacologically acceptable carrier substances, auxiliary substances and/or vehicles.

83. The preparation or supplement of claim 82, wherein the preparation or supplement comprises a substantially saponine-free ethanol-insoluble component of an aqueous or hydroalcoholic raw extract.

84. The preparation or supplement of claim 82, wherein the preparation or supplement is present as a vegetable diuretic preparation.

85. A method of promoting a diuresis, of promoting a purification of the body, of reducing body weight, of preventing or treating heart failure, of preventing or treating hypertension and/or of preventing or treating heart hypertrophia in a subject in need thereof, wherein the method comprises administering to the subject an effective amount of one or more extracts according to claim 50.
